(19) United States
(12) Patent Application Publication
(10) Pub. No.: US 2015/0157239 A1
(43) Pub. Date: Jun. 11, 2015

(54) Cardiovascular and Pulmonary Radar System

(71) Applicants: Daniel J. Rissacher, Potsdam, NY (US); Stephanie A. Schuckers, Canton, NY (US); William D. Jemison, Potsdam, NY (US)

(72) Inventors: Daniel J. Rissacher, Potsdam, NY (US); Stephanie A. Schuckers, Canton, NY (US); William D. Jemison, Potsdam, NY (US)

(73) Assignee: CLARKSON UNIVERSITY, Potsdam, NY (US)

(21) Appl. No.: 14/563,738

(22) Filed: Dec. 8, 2014

Related U.S. Application Data
(60) Provisional application No. 61/912,920, filed on Dec. 6, 2013.

(51) Int. Cl. A61B 5/05; A61B 5/08; A61B 5/0205; A61B 5/0452; A61B 7/04

(52) U.S. Cl. CPC A61B 5/0507; A61B 5/0452; A61B 7/04; A61B 5/0205; A61B 5/08

(57) ABSTRACT

Systems and methods for monitoring a physiological parameter of a person using radar. The radar can be configured to obtain a radar return signal, the radar return signal received through a receive antenna and including a first physiological signal from the person. The system can also include a physiological signal sensor device, such as an ECG, comprising a sensor configured to obtain a second physiological signal from the person. A computer receives the first and second physiological signals and uses them to produce a time-locked average radar signal, the time-locked average radar signal including a physiological parameter of the person with reduced noise.

### CROSS-REFERENCE TO RELATED APPLICATIONS

This application claims priority to U.S. Provisional Patent Application Ser. No. 61/912,920, filed on Dec. 6, 2013, and entitled “Cardiovascular and Pulmonary Radar System,” the entire disclosure of which is incorporated herein by reference.

### STATEMENT REGARDING FEDERALLY SPONSORED RESEARCH OR DEVELOPMENT

This invention was made with government support under Contract No. 12F-21C-12 awarded by the National Science Foundation. The government has certain rights in the invention.

### BACKGROUND

The present disclosure relates to methods and systems for radar detection and monitoring of physiological signals, and more particularly, to methods and systems for remote radar detection and monitoring of physiological signals for subject identification and diagnostics.

Doppler radar has been utilized for physiological monitoring of cardiopulmonary signals since the 1970's. These radar systems have been suggested for physiological analysis of the heart, with theoretical and experimental work demonstrating that markers in the radar waveform, detected by the radar system, can be correlated to different stages of the heart contraction cycle. However, these radar systems have traditionally been large and inefficient, and are subject to considerable background noise that interferes with detection and interpretation of the waveform.

It is also known that cardiac patterns and other biometrics vary from person to person, and thus this data can be used for identification of an individual whose patterns/biometrics are known. Indeed, electrocardiogram (“ECG”), phonocardiogram (“PCG”), and laser Doppler velocimetry (“LDV”) data have all been successfully used as metrics for biometric identification. However, these systems require direct physical contact (such as electrodes) or a laser contact with the individual to be identified.

Accordingly, there is a continued need in the art for systems and methods for remote detection and monitoring of physiological signals for subject identification and diagnostics.

### SUMMARY OF THE INVENTION

The present disclosure is directed to inventive methods and apparatus for a radar system. Various embodiments and implementations herein are directed to a radar system. A primary objective of the radar system is to collect thoracic data that can be used for clinical diagnostics. A further objective of the radar system is to collect thoracic data that can be used for biometrics to allow for subject identification. According to various embodiments, the radar system includes a single antenna (i.e., monostatic system) to transmit and receive radar energy from the subject. The subject-antenna distance and orientation can be varied to provide unique desired metrics. According to another embodiment, the radar system includes two antennas (i.e., bistatic system), or more than two antennas to transmit and receive radar energy from the subject. The subject-antenna distance and orientation can be varied to provide unique desired metrics. The multiple antennas allow either reflected energy to be received, or transmitted in the case for example with a transmit antenna is in front of the subject and the receive is behind the subject.

Another objective of the radar system is to simultaneously collect other cardiac or pulmonary measures to facilitate radar data processing. For instance one cardiac measure is the electrocardiogram (ECG). The ECG can be used as a time reference to produce a time-locked average radar signal, producing an average heartbeat over many cardiac cycles. A time-locked average signal is also known as an ensemble average or Spike Triggered Average (“STA”). The averaging technique allows optimization of cardiac radar signal-to-noise ratio.

Generally in one aspect, a radar system includes: (i) a radar configured to obtain a radar return signal, the radar return signal received through a receive antenna and comprising a first physiological signal from the person; (ii) a physiological signal sensor device comprising a sensor configured to obtain a second physiological signal from the person; and (ii) a computer configured to produce, using the first and second physiological signals, a time-locked average radar signal, where the time-locked average radar signal comprises the physiological parameter of the person.

According to an embodiment, the radar comprises at least two separate antennas, a transmit antenna and a receive antenna. According to an embodiment, the receive antenna and the user are spaced at a predetermined distance.

According to an embodiment, the second physiological signal is electrical activity of the person's heart.

According to an embodiment, the physiological signal sensor device is an electrocardiogram.

According to an embodiment, the physiological signal sensor device is a phonocardiogram.

According to an embodiment, the second physiological signal is respiration of the person.

Generally, in one aspect, a method for monitoring a physiological parameter of a person is provided. The method includes the steps of: (i) obtaining, using a radar system, a radar return signal comprising a first physiological signal from the person; (ii) obtaining, using a physiological signal sensor device, a second physiological signal from the person; and (iii) producing a time-locked average radar signal using the first and second physiological signals, where the time-locked average radar signal comprises the physiological parameter of the person.

According to an embodiment, the method includes the steps of demodulating the radar return signal, and extracting the first physiological signal.

According to an embodiment, the method includes the steps of identifying one or more peaks in the second physiological signal over time, and utilizing the one or more peaks as a time-reference.

According to an embodiment, the method includes the step of averaging the first physiological signal over a window of time before and after each of the one or more peaks, the window comprising a predetermined amount of time, to produce the time-locked average radar signal.

According to an embodiment, the method includes the step of performing a spectrogram transformation of the time-locked average radar signal.

According to an embodiment, the method includes the step of performing frequency or time-frequency analysis (e.g. wavelet transform) of the time-locked average radar signal.

Generally, in one aspect, a method for attempting to identify a person is provided. The method includes the steps of: (i) obtaining, using a radar system, a radar return signal comprising a first physiological signal from the person; (ii) obtaining, using a physiological signal sensor device, a second physiological signal from the person; (iii) producing a time-locked average radar signal using the first and second physiological signals, wherein the time-locked average radar signal comprises a physiological parameter of the person; and (iv) comparing the physiological parameter to a database of physiological parameters.

According to an embodiment, the method includes the step of finding a close match between the person's physiological parameter and a physiological parameter in the database.

According to an embodiment, the method includes the step of authenticating an individual based on similarity to a template or identification of an individual based on similarity to a database of templates.

According to an embodiment, the method includes the steps of demodulating the radar return signal, and extracting the first physiological signal.

According to an embodiment, the method includes the steps of identifying one or more peaks in the second physiological signal over time, and utilizing the one or more peaks as a time-reference.

According to an embodiment, the method includes the step of averaging the first physiological signal over a window of time before and after each of the one or more peaks, the window comprising a predetermined amount of time, to produce the time-locked average radar signal.

According to an embodiment, the method includes the step of performing a spectrogram transformation of said time-locked average radar signal.

According to an embodiment, the method includes the step of performing a wavelet transformation of said time-locked average radar signal.

### BRIEF DESCRIPTION OF THE DRAWINGS

The present invention will be more fully understood and appreciated by reading the following Detailed Description in conjunction with the accompanying drawings, in which:

FIG. 1 is a schematic representation of a radar system for physiological signal detection in accordance with an embodiment.

FIGS. 2A and 2B are schematic representations of a monostatic (FIG. 2A) antenna configuration and a bistatic (FIG. 2B) antenna configuration of a radar system in accordance with an embodiment.

FIG. 3 is graph of electrocardiogram (“ECG”), phonocardiogram (“PCG”), and radar data collected from an individual in accordance with an embodiment.

FIG. 4 is a graph of an averaged radar signal over multiple heartbeats time-locked to the R-wave of an ECG, in accordance with an embodiment.

FIG. 5 is a flowchart depicting a method for physiological signal detection in accordance with an embodiment.

### DETAILED DESCRIPTION

The present disclosure describes various embodiments of apparatus, systems, devices, and methods for a radar system and method. One goal of the embodiments of the present disclosure is to provide a radar system for the remote detection and monitoring of physiological signals, to be utilized in subject identification and diagnostics. In view of the foregoing, various embodiments and implementations are directed to a radar system in which a radar includes a monostatic or bistatic antenna configuration for the detection of physiological signals, as well as a novel computer system for interpreting the received signals.

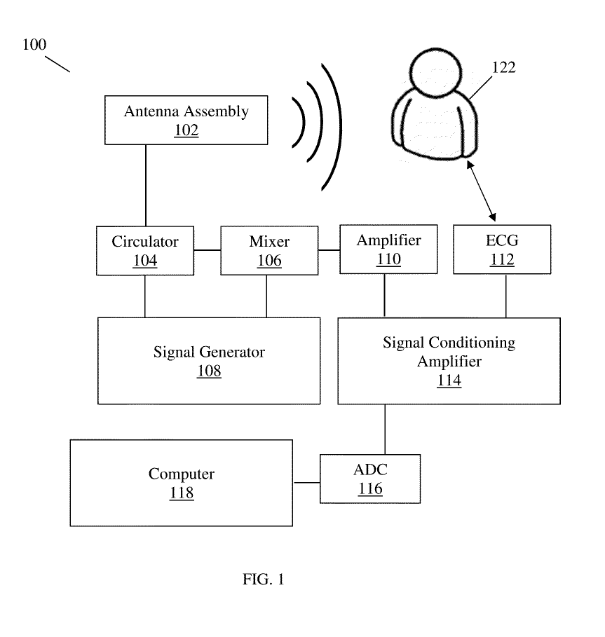

Referring to FIG. 1, in one embodiment, a radar system 100 is provided for analysis of physiological signals. For example, according to an embodiment, radar system 100 can be used without, or in conjunction with, one or more signals from a direct physiological connection to the user, including ECG, PCG, and/or LDV. Radar system 100 comprises an antenna assembly 102. Antenna assembly 102 can include a single antenna or multiple antennas. The radar system 100 can also include a circulator 104, a mixer 106, signal generator 108, amplifier 110, signal conditioning amplifier 114, analog-to-digital converter 116, and computer 118.

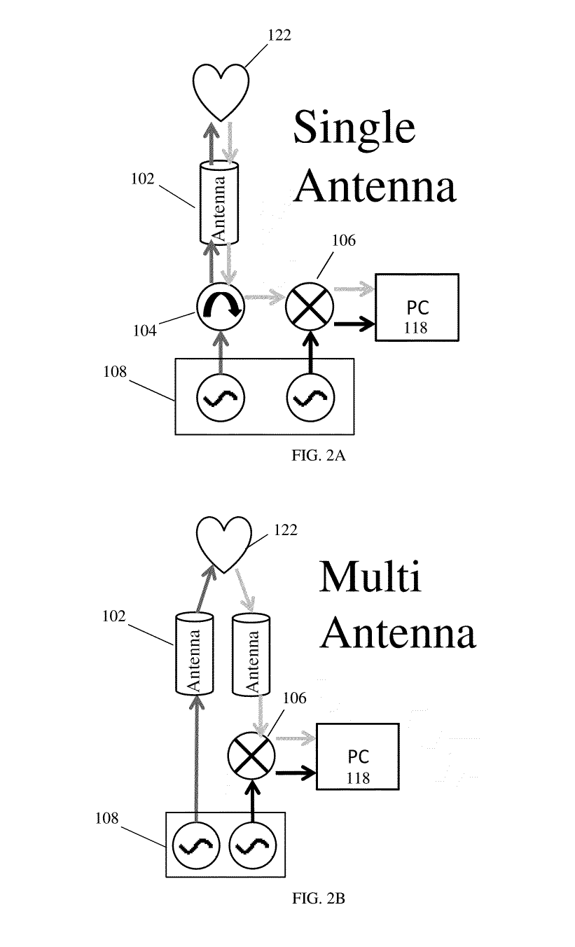

Referring to FIGS. 2A and 2B, in various embodiments, are radio frequency components and signal flow in a monostatic (FIG. 2A) and bistatic (FIG. 2B) configuration of radar system 100. In the monostatic configuration, two signals are produced by signal generator 108, a transmit frequency and an intermediate frequency for modulation. The transmit frequency is sent to circulator 104 which directs the signal to the monostatic antenna assembly 102 which then transmits the radio frequency towards the subject. In this monostatic configuration, the signal is reflected from the subject 122 and then received by the same antenna and back to circulator 104 which directs it to mixer 106 which combines the received signal and the intermediate frequency to be processed on computer 118 after data acquisition.

In the bistatic configuration of radar system 100, shown in FIG. 2B, the signal transmit frequency is connected to the transmit antenna and a separate antenna is used to receive the signal after it is reflected from, or transmitted through, the subject. The transmit antenna and receive antenna are part of antenna assembly 102, and can be associated or placed in different locations. The received signal is combined with the intermediate frequency by mixer 106 and digitized on computer 118.

Radar system 100 can be configured to simultaneously collect additional cardiac or pulmonary measures or signals, thereby facilitating radar data processing. For instance, radar system 100 in FIG. 1 includes physiological signal sensor device 112 configured to collect data from user 122. In this embodiment, physiological signal sensor device 112 is an ECG apparatus. The can be used as a time reference to produce a time-locked average radar signal, producing an average heartbeat over many cardiac cycles. This averaging technique allows optimization of cardiac radar signal-to-noise ratio. In addition to cardiac or pulmonary measures or signals, other physiological measurements, characteristics, and/or signals could be obtained. For example, in various embodiments, the physiological signal sensor device 112 may be a ballistocardiogram (“BCG”), seismocardiogram (“SCG”), laser doppler velocimetry (“LDV”) device, pulse oximeter (or SpO₂ or IR pulse sensor), an ultrasound, or a variety of other physiological signal sensor devices configured or adapted to collect a physiological signal from a user.

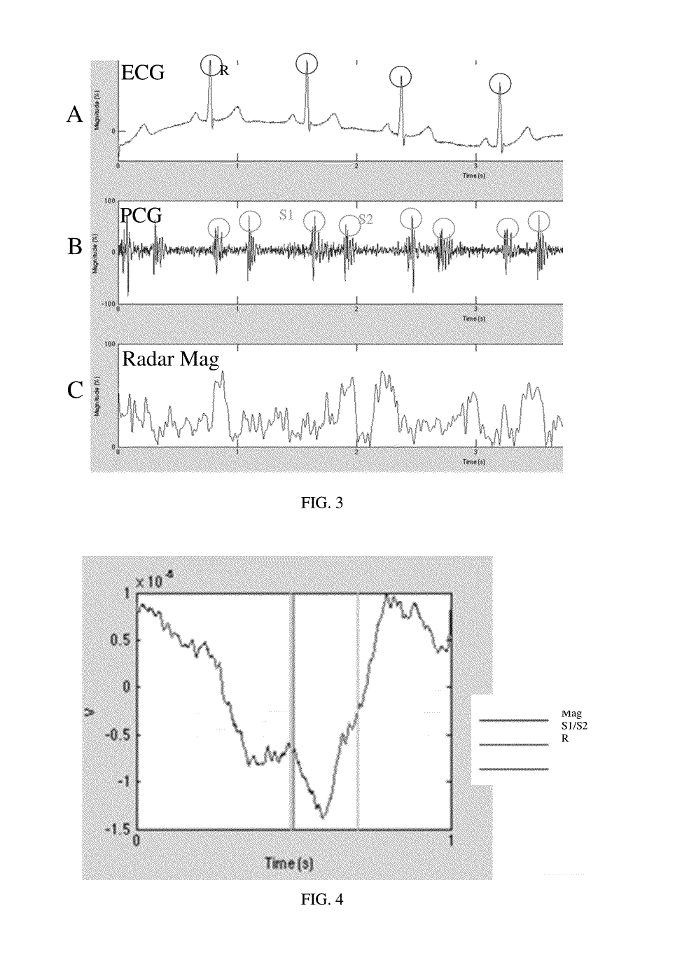

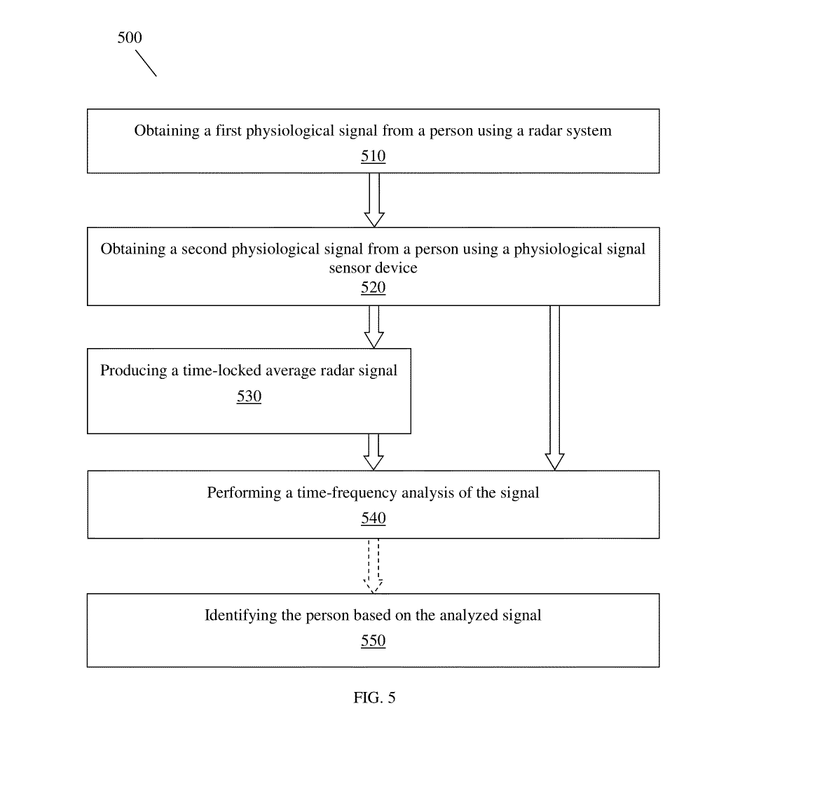

Referring to FIGS. 3 and 4, in one embodiment, are depictions of the cardiac referenced time-lock average. The figures demonstrate the signal received by radar system 100 (FIG. 3C), and that two cardiac metrics, electrocardiogram (ECG, with R-wave peak shown, FIG. 3A) and phonocardiogram (PCG, with sounds S1 and S2 shown, FIG. 3B) can be used as a time reference. For example, the system can simultaneously acquire electrocardiogram (ECG or EKG) and radar signals, and can utilize the ECG R-wave as a time reference allowing the computation of the average radar signal over multiple heartbeats time-locked to the R-wave. Indeed, the ECG or other signals can be used to average the radar signal over many heartbeats to obtain an average heartbeat signal time-locked to a cardiac event (e.g., ECG R-Wave), resulting in a spike-triggered average (“STA”) shown in FIG. 4. This allows removal of non-cardiac information (such as respiratory signals) and maximization of cardiac signal-to-noise ratio as shown in the single trial radar magnitude and mean beat radar magnitude.

According to an embodiment, radar system 100 includes signal generator 108 producing a 2.4 GHz continuous wave output signal and a 1 kHz intermediate frequency, although many other frequencies are possible. The output signal is directed through antenna 102 toward subject 122, and the return signal is collected through either the same antenna (monostatic configuration) or an additional antenna (bistatic configuration). The returned signal is then modulated by the intermediate frequency signal and digitized. The digital signal is then demodulated and cardiac or respiratory signals of interest are extracted. The bistatic configuration facilitates a variety of transmit-antenna/subject/receive-antenna orientations that each yield unique physiological data.

In accordance with an embodiment, the signal received from the radar system is processed by computer 118 using one or more algorithms or filters, which extract the cardiac signal. For example, the signal can undergo IQ demodulation, a low-pass filter using a frequency below the modulation frequency, and more. For example, this processing can remove low frequency signals, such as respiratory signals, and high frequency noise.

The extracted cardiac signal can then be utilized to find the average heartbeat (or other physiological signal), or the STA. For example, one or more R-wave peaks are detected from the ECG (or other input) as a heartbeat time-reference, and the radar data is averaged for a predetermined window before and after the R-wave peak.

STAs like the one depicted in FIG. 4 enable the use of other downstream processing such as spectrogram and wavelet transformations. These transformations, for example, allow the data to be compared to data obtained from other subjects, or to databases of similar data. The peaks of the spectrogram transformed data, for example, can be compared to other subject's peaks. As an example of this comparison, the data can be searched point by point with distances between the points determined. The wavelet transformation, such as a Continuous Wavelet Transform (CWT), can include an algorithm that finds clusters and centroids in the data for comparisons. According to yet another embodiment, the symmetric relative entropy (SRE) score can be determined for one or more subjects using the processed data.

In addition to time-frequency analysis, other types of analysis can be performed. For example, there can be a strictly time-domain analysis (e.g., correlation), and/or a frequency-domain analysis (e.g., fourier transform). Each of these analyses, as well as the time-frequency analysis, can be used with or without time-locked averaging described above.

According to one embodiment, therefore, the radar and additional information (such as ECG) is processed by the following steps:

1. Obtain radar and the additional accompanying data (such as ECG);

2. Demodulate samples into I and Q;

3. Identify peaks (such as heartbeat peaks, respiratory peaks, etc.) from the additional accompanying data;

4. Perform a spectrogram transformation of the sampled data;

5. Perform a wavelet transformation of the sampled data;

6. Find peaks for the spectrogram and/or wavelet transformations; and

7. Compare the peaks and perform identification or other analysis.

To perform identification, for example, the processed physiological signals—which presumably will be substantially unique to the individual—can be compared to a database of previously recorded physiological signals to determine if there are any matches. If there are no perfect matches, a best fit or other close matching system or algorithm can be utilized.

According to an embodiment, the radar system 100 can be utilized to determine and analyze not only normal physiological signals, but also to determine and analyze physiological signals from subjects with cardiac conditions and/or defects.

Referring to FIG. 5, in accordance with an embodiment, is a method 500 for monitoring a physiological parameter of a person. At step 510 of the method, a radar system is utilized to obtain a radar return signal comprising a first physiological signal from the person. The radar system can be any of the systems described or otherwise envisioned herein. The first physiological signal can be, for example, cardiac, respiratory, brain, or other signals.

At step 520 of the method, a physiological signal sensor device is utilized to obtain a second physiological signal from the person. The physiological signal sensor device 112 may be an ECG, PCG, BCG, SCG, LDV device, pulse oximeter, SpO₂ or IR pulse sensor, an ultrasound, or a variety of other physiological signal sensor devices configured or adapted to collect a physiological signal from a user. The second physiological signal can be, for example, cardiac, respiratory, brain, or other signals.

At step 530 of the method, a computer receives the signals and produces a time-locked average radar signal using the first and second physiological signals, wherein the time-locked average radar signal includes or is the physiological parameter, characteristic, or signal of the person. Producing a time-locked average radar signal can include, among other things, demodulation of signals into I and Q, filtering, and extraction of the first physiological signal from the radar receive, identification of peaks (such as heartbeat peaks, respiratory peaks, etc.) from the additional accompanying data obtained from the physiological signal sensor device, and/or identifying one or more peaks in the second physiological signal over time and utilizing the one or more peaks as a time-reference.

At step 540, which can proceed directly from step 520 or can skip over step 530, a frequency or time-frequency analysis (e.g. wavelet transform) of the time-locked average radar signal is performed. The analysis allows the data to be compared to data obtained from other subjects, or to databases of similar data. A wavelet transformation, such as a CWT, can include an algorithm that finds clusters and centroids in the data for comparisons.

At step 550, which is optional, the obtained data can be utilized to identify or authenticate an individual. For example, the determined physiological parameter of the person (determined using the first and second physiological signals) can be compared to a database of physiological parameters to find a match or similarity. For example, the database can be searched to find a close match between the person's physiological parameter and a physiological parameter in the database. The identification and authentication rely on a match or similarity being identified between the obtained data and a profile or template stored in the database.

Although the present invention has been described in connection with a preferred embodiment, it should be understood that modifications, alterations, and additions can be made to the invention without departing from the scope of the invention as defined by the claims.

What is claimed is:

1. A system for monitoring a physiological parameter of a person, the system comprising:
a radar configured to obtain a radar return signal, the radar return signal received through a receive antenna and comprising a first physiological signal from the person;
a physiological signal sensor device comprising a sensor configured to obtain a second physiological signal from the person; and
a computer configured to produce, using the first and second physiological signals, a time-locked average radar signal;
wherein the time-locked average radar signal comprises the physiological parameter of the person.

2. The system of claim 1, wherein the radar comprises at least two separate antennas, a transmit antenna and a receive antenna.

3. The system of claim 1, wherein the receive antenna and the user are spaced at a predetermined distance.

4. The system of claim 1, wherein the second physiological signal is electrical activity of the person's heart.

5. The system of claim 4, wherein the physiological signal sensor device is an electrocardiogram.

6. The system of claim 1, wherein the physiological signal sensor device is a phonocardiogram.

7. The system of claim 1, wherein the second physiological signal is respiration of the person.

8. A method for monitoring a physiological parameter of a person, the method comprising the steps of:
obtaining, using a radar system, a radar return signal comprising a first physiological signal from the person;
obtaining, using a physiological signal sensor device, a second physiological signal from the person; and
producing a time-locked average radar signal using the first and second physiological signals, wherein the time-locked average radar signal comprises the physiological parameter of the person.

9. The method of claim 8, wherein said producing step comprises the steps of:
demodulating the radar return signal; and
extracting the first physiological signal.

10. The method of claim 8 wherein said producing step comprises the steps of:
identifying one or more peaks in the second physiological signal over time; and
utilizing the one or more peaks as a time-reference.

11. The method of claim 10, further comprising the step of averaging the first physiological signal over a window of time before and after each of the one or more peaks, the window comprising a predetermined amount of time, to produce the time-locked average radar signal.

12. The method of claim 11, further comprising the step of performing a spectrogram transformation of said time-locked average radar signal.

13. The method of claim 11, further comprising the step of performing a frequency or time-frequency analysis of said time-locked average radar signal.

14. A method for attempting to identify or authenticate a person, the method comprising the steps of:
obtaining, using a radar system, a radar return signal comprising a first physiological signal from the person;
obtaining, using a physiological signal sensor device, a second physiological signal from the person;
producing a time-locked average radar signal using the first and second physiological signals, wherein the time-locked average radar signal comprises a physiological parameter of the person; and
comparing the physiological parameter to a database of physiological parameters.

15. The method of claim 14, further comprising the step of finding a close match between the person's physiological parameter and a physiological parameter in the database.

16. The method of claim 14, further comprising the step of identifying or authenticating the person based on a close match between the person's physiological parameter and a template in the database.

17. The method of claim 14, wherein said producing step comprises the steps of:
demodulating the radar return signal; and
extracting the first physiological signal.

18. The method of claim 14 wherein said producing step comprises the steps of:
identifying one or more peaks in the second physiological signal over time; and
utilizing the one or more peaks as a time-reference.

19. The method of claim 18, further comprising the step of averaging the first physiological signal over a window of time before and after each of the one or more peaks, the window comprising a predetermined amount of time, to produce the time-locked average radar signal.

20. The method of claim 19, further comprising the step of performing a spectrogram transformation of said time-locked average radar signal.
